(19) United States
(12) Patent Application Publication
(10) Pub. No.: US 2024/0008931 A1
(43) Pub. Date: Jan. 11, 2024

(54) SURGICAL SYSTEM AND METHOD OF OPERATING SURGICAL SYSTEM

(71) Applicants: MEDICAROID CORPORATION, Kobe-shi (JP); KAWASAKI JUKOGYO KABUSHIKI KAISHA, Kobe-shi (JP)

(72) Inventors: Masakazu TAKAHASHI, Kobe-shi (JP); Shunya YOSHISE, Osaka-shi (JP); Yuji KISHIDA, Kobe-shi (JP)

(73) Assignees: MEDICAROID CORPORATION, Kobe-shi (JP); KAWASAKI JUKOGYO KABUSHIKI KAISHA, Kobe-shi (JP)

(21) Appl. No.: 18/348,379

(22) Filed: Jul. 7, 2023

(30) Foreign Application Priority Data
Jul. 11, 2022 (JP) 2022-111405

(51) Int. Cl. A61B 34/20; A61B 34/32

(52) U.S. Cl. CPC A61B 34/20; A61B 34/32; A61B 2034/2057; A61B 2034/301

(57) ABSTRACT

A surgical system according to one or more embodiments may include: a first manipulator supporting an endoscope; a second manipulator supporting a surgical instrument; an operating device configured to receive a command to operate the surgical instrument; and a controller configured to operate, when a distal end of the surgical instrument is located in the field of view of the endoscope, the surgical instrument in a first operation mode that controls the operation of the surgical instrument according to the command, and to change, when the distal end of the surgical instrument is located outside the field of view of the endoscope, the operation of the surgical instrument from the first operation mode to a second operation mode.

This application claims priority based on 35 USC 119 from prior Japanese Patent Application No. 2022-111405 filed on Jul. 11, 2022, entitled “SURGICAL SYSTEM AND METHOD OF OPERATING SURGICAL SYSTEM”, the entire contents of which are incorporated herein by reference.

### BACKGROUND

The disclosure may relate to a surgical system and a method of operating a surgical system.

In a related art, there is known a surgical system (for example, Patent Document 1: Japanese Patent Application Publication No. 2013-188574)

Patent Document 1 discloses a robotic surgical system including manipulators supporting an endoscope and tools (surgical instruments) respectively, an P device configured to receive commands to operate the tools, and a controller configured to control operation of the tools. In the surgical system disclosed in Patent Document 1, the controller is configured to determine whether or not a distal end of the tool (surgical instrument) to be operated by the operating device is located outside of a field of view of the endoscope, and display information of the tool located outside the field of view as a symbol (a sign) in a border area outside a display area of a screen of a monitor.
Patent Document 1: Japanese Patent Application Publication No. 2013-188574

### SUMMARY

In such a surgical system disclosed in Patent Document 1, even if the distal end of the tool is located outside the field of view of the endoscope, a doctor can manipulate the tool outside of the field of view with the operating device in the same way as in the tool is located within the field of view. Accordingly, in the state where the distal end of the tool (the surgical instrument) is located outside the field of view of the endoscope and thus the distal end of the tool cannot be visually checked by the doctor, the distal end of the tool that is located outside the field of view of the endoscope might unintentionally come in contact with an object.

An object of one or more embodiments of the disclosure may be to provide a surgical system and a method of operating a surgical system that are capable of preventing a surgical instrument outside a field of view of an endoscope from unintentionally coming in contact with an object.

A first aspect of one or more embodiments of the disclosure may be a surgical system that may include: a first manipulator that supports an endoscope; a second manipulator that supports a surgical instrument; an operating device configured to receive a command to operate the surgical instrument; and a controller including one or more processors and configured to control operation of the surgical instrument in a first operation mode that controls the operation of the surgical instrument in response to the command received through the operating device. The controller is configured to determine, based on a field of view of the endoscope and a position of a distal end of the surgical instrument, whether or not the distal end of the surgical instrument is located outside the field of view of the endoscope, when it is determined that the distal end of the surgical instrument is located inside the field of view of the endoscope, control the operation of the surgical instrument in the first operation mode, and when the distal end of the surgical instrument is located outside the field of view of the endoscope, change the operation of the surgical instrument from the first operation mode to a second operation mode.

According to the first aspect as described above, the controller determines, based on the field of view of the endoscope and the position of the distal end of the surgical instrument, whether or not the distal end of the surgical instrument is located outside the field of view of the endoscope, and changes, when it is determined that the distal end of the surgical instrument is located outside the field of view of the endoscope, the operation of the surgical instrument from the first operation mode that controls the operation of the surgical instrument in response to the command received through the operating device to the second operation mode. Accordingly, when the distal end of the surgical instrument is located outside the field of view of the endoscope and thus the operator cannot visually confirm the distal end of the surgical instrument, it is possible restrict the operation of the surgical instrument such as not to operate the surgical instrument, and thereby suppressing the surgical instrument from unintentionally coming in contact with an object such as a patient's body or the like.

A second aspect of one or more embodiments of the disclosure may be a method of operating a surgical instrument by a controller in a surgical system that may include: determining, based on a relationship between a field of view of an endoscope that is supported by a first manipulator and a position of a distal end of a surgical instrument that is supported by a second manipulator, whether or not the distal end of the surgical instrument is located outside the field of view of the endoscope; when it is determined that the distal end of the surgical instrument is located inside the field of view of the endoscope, controlling operation of the surgical instrument in a first operation mode that controls the operation of the surgical instrument in response to the command received through the operating device; and when it is determined that the distal end of the surgical instrument is located outside the field of view of the endoscope, changing the operation of the surgical instrument from the first operation mode to a second operation mode, and controlling the operation of the surgical instrument in the second operation mode.

According to the second aspect described above, when the distal end of the surgical instrument is located outside the field of view of the endoscope, the operation of the surgical instrument is changed from the first operation mode for the state where the distal end of the surgical instrument is located in the field of view of the endoscope to the second operation mode for the state where the distal end of the surgical instrument is located outside the field of view of the endoscope. Accordingly, when the distal end of the surgical instrument is located outside the field of view of the endoscope and thus the operator cannot visually confirm the distal end of the surgical instrument, it is possible restrict the operation of the surgical instrument such as not to operate the surgical instrument, and thereby suppressing the surgical instrument from unintentionally coming in contact with an object such as a patient's body or the like.

According to at least one of the aspects described above, it is possible to suppress the surgical instrument from unintentionally coming in contact with an object such as a patient's body or the like.

### BRIEF DESCRIPTION OF DRAWINGS

FIG. 1 is a diagram illustrating a view of a configuration of a surgical system according to an embodiment;

FIG. 2 is a diagram illustrating a view of a configuration of a medical manipulator according to an embodiment;

FIG. 3 is a diagram illustrating a view of a configuration of an input device according to an embodiment;

FIG. 4 is a diagram illustrating a view of a configuration of an operation handle according to an embodiment;

FIG. 5 is a diagram illustrating a view of foot pedals according to an embodiment;

FIG. 6 is a diagram illustrating a view of a configuration of an arm of the medical manipulator according to an embodiment.

FIG. 7 is a diagram illustrating a view of forceps;

FIG. 8 is a diagram illustrating a perspective view of a configuration of an operation unit of the medical manipulator according to an embodiment;

FIG. 9 is a diagram illustrating a view of an endoscope;

FIG. 10 is a diagram illustrating a view of a pivot position setting device;

FIG. 11 is a block diagram of a configuration of a control unit of the medical manipulator according to an embodiment;

FIG. 12 is a diagram illustrating an image captured by the endoscope and a graphical user interface.

FIG. 13 is a diagram illustrating a view of the graphical user interface including a plurality of areas;

FIG. 14 is a diagram for explaining setting of an operation change mode through a touch panel of a remote control apparatus;

FIG. 15 is a diagram for explaining selection of a surgical site through an input device of a medical cart;

FIG. 16 is a diagram for explaining examples of a mark according to an embodiment.

### DETAILED DESCRIPTION

Descriptions are provided hereinbelow for one or more embodiments based on the drawings. In the respective drawings referenced herein, the same constituents are designated by the same reference numerals and duplicate explanation concerning the same constituents is omitted. All of the drawings are provided to illustrate the respective examples only.

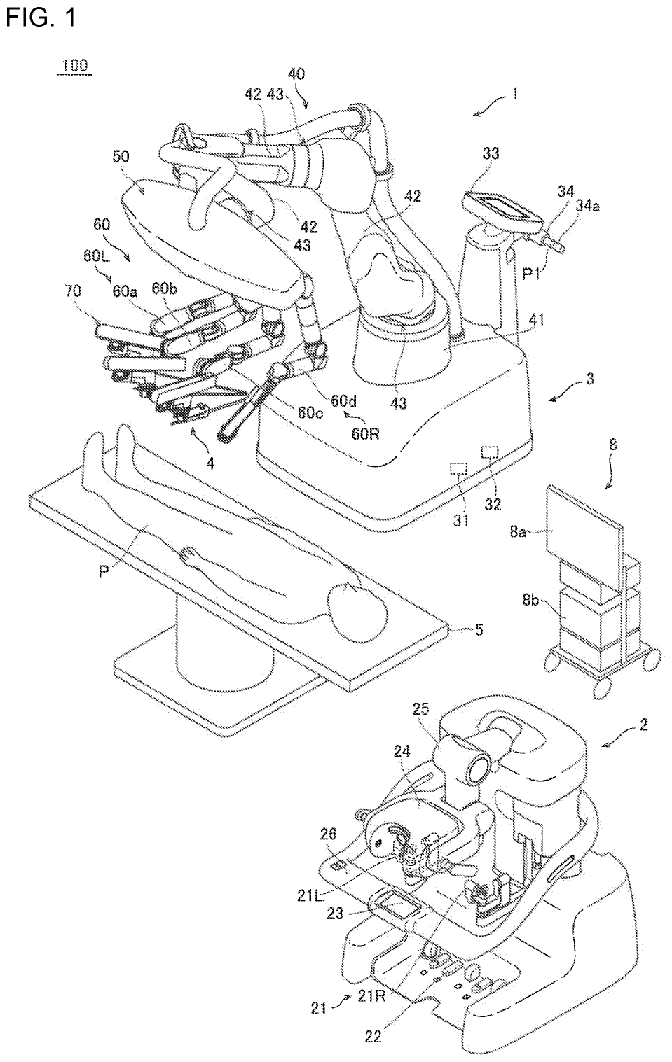

With reference to FIGS. 1 to 16, a configuration of a surgical operation system 100 according to an embodiment is described below. The surgical operation system 100 includes a medical manipulator 1 serving as a patient-side apparatus and a remote control apparatus 2 serving as an operator-side apparatus to operate the medical manipulator 1. The medical manipulator 1 is provided with a medical trolley 3 and is thus configured to be movable. The remote control apparatus 2 is provided at a location away from the medical manipulator 1. The medical manipulator 1 is configured to be remotely operated by the remote control apparatus 2. An operator (such as a doctor) inputs to the remote control apparatus 2 a command (an instruction) that causes the medical manipulator 1 to perform a desired operation. The remote control apparatus 2 transmits the input command to the medical manipulator 1. The medical manipulator 1 operates in response to the command received. The medical manipulator 1 is disposed in a surgery room, as a sterile field, which is sterilized. The surgical operation system 100 is an example of a “surgical system.”

The remote control apparatus 2 is arranged independently from the medical manipulator 1. The remote control apparatus 2 is disposed inside the surgery room or outside the surgery room, for example. The remote control apparatus 2 accepts commands (instructions) inputted by an operator for operating the medical instrument 4. The remote control apparatus 2 includes operation handles 21, foot pedals 22, a touch panel 23, a monitor 24, a support arm 25, and a support bar 26. The operation handles 21 are hand controllers (HC) provided for the operator (such as a doctor) to input commands. Note that the operation handles 21 and the foot pedals 22 are examples of “operating devices.” The touch panel 23 is an example of a “mode setting unit” or an “user interface.” The monitor 24 is an example of a “display device.”

The operation handles 21 are configured to operate the medical instruments 4. Specifically, the operation handles 21 receive an amount of movement inputted by the operator O to operate the medical instruments 4. The operation handles 21 include an operation handle 21L, which is arranged on the left side of the operator (such as a doctor) and is to be operated by the left hand of the operator O, and an operation handle 21R, which is arranged on the right side of the operator and is to be operated by the right hand of the operator O.

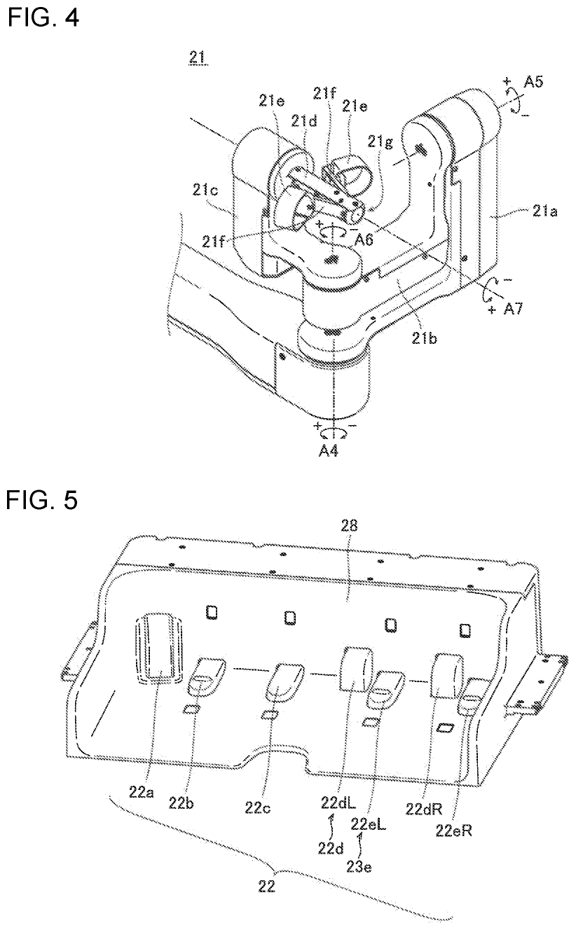

As illustrated in FIG. 4, each of the operation handles 21 includes a link portion 21a, a link portion 21b, a link portion 21c, and a link portion 21d that is to be operated by the operator (such as a doctor). The link portion 21a is rotatable about an axis (joint) A4. By rotating the link portion 21a around the axis A4, an arm portion 61 described later rotates about an axis (joint) JT4. The link portion 21b is rotatable about an axis (joint) A5 with respect to the link portion 21a. By rotating the link portion 21b around the axis A5, the arm portion 61 described later rotates about an axis (joint) JT5. The link portion 21c is rotatable about an axis (joint) A6 with respect to the link portion 21b. By rotating the link portion 21c around the axis A6, the arm portion 61 rotates about an axis (joint) JT6. The link portion 21d is rotatable about an axis (joint) A7 with respect to the link portion 21c. By rotating the link portion 21d around the axis A7, the arm portion 61 rotates about an axis (joint) JT7. Note that the medical instrument 4 is an example of a surgical instrument.

The operation handle 21 includes a pair of grip members 21f to be opened or closed by the operator. Each grip member 21f is formed of a lever member having an elongated plate shape. The pair of grip members 21f each includes a proximal end which is rotatably connected to a proximal end 21g of the link portion 21d. The operator inserts fingers into the pair of finger insertion portions 21e to operate the operation handle 21. The operator inserts fingers into the pair of finger insertion portions 21e and operates the operation handle 21. A proximal end of each of the pair of grip members 21f is connected to the link portion 21d. By increasing or decreasing the angle between the pair of grip members 21f, the opening angle between a first jaw member 104a and a second jaw member 104b is changed.

Further, a movement amount of the arm 60 (medical instrument 4) is scaled (changed) with respect to the operation amount received by the operation handle 21. That is, the medical instrument 4 is set to move at a predetermined scale in response to the movement command. For example, when the movement scaling ratio is set to ½, the medical instrument 4 moves ½ of the movement distance of the operation handle 21. This allows for precise fine surgery.

As illustrated in FIG. 5, the plural foot pedals 22 are provided to execute functions of the medical instrument 4. The plural foot pedals 22 are arranged on a base 28. The foot pedals 22 include a switch pedal 22a, a clutch pedal 22b, a camera pedal 22c, cutting pedals 22d, and coagulation pedals 22e. The switch pedal 22a, the clutch pedal 22b, the camera pedal 22c, the cutting pedals 22d, and the coagulation pedals 22e are operated by the foot of the operator. The cutting pedals 22d include a cutting pedal 22dR for the right arm 60 and a cutting pedal 22dL for the left arm 60. The coagulation pedals 22e include a coagulation pedal 22eR for the right arm 60 and a coagulation pedal 22eL for the left arm 60.

The switch pedal 22a is configured to select one of the arms 60 that is to be operated by the operation handles 21. The clutch pedal 22b is configured to perform a clutch operation that temporarily disconnects an operational connection (a control-related connection) between the arm 60 and the operation handle 21. While the clutch pedal 22b is depressed by the operator, the operation by the operation handle 21 is not transmitted to the arm 60.

The camera pedal 22c is operated for allowing the two operation handles 21 to move the endoscope 6. Specifically, the camera pedal 22c is provided for inputting a command that enables the endoscope 6 to be moved. Specifically, in response to the camera pedal 22c being depressed (stepped) by the operator, the command that enables the endoscope 6 to be moved is inputted. That is, while the command that enables the endoscope 6 to move is being inputted by the camera pedal 22c (that is, while the camera pedal 22c is depressed by the operator), the endoscope 6 is able to be moved by moving both of the operation handle 21R and operation handle 21L.

While the cutting pedal 22d (coagulation pedal 22e) is depressed by the operator, a high-frequency current for incising or coagulating tissue flows from an electrosurgical device (not illustrated) to the medical instrument 4 (electrocautery).

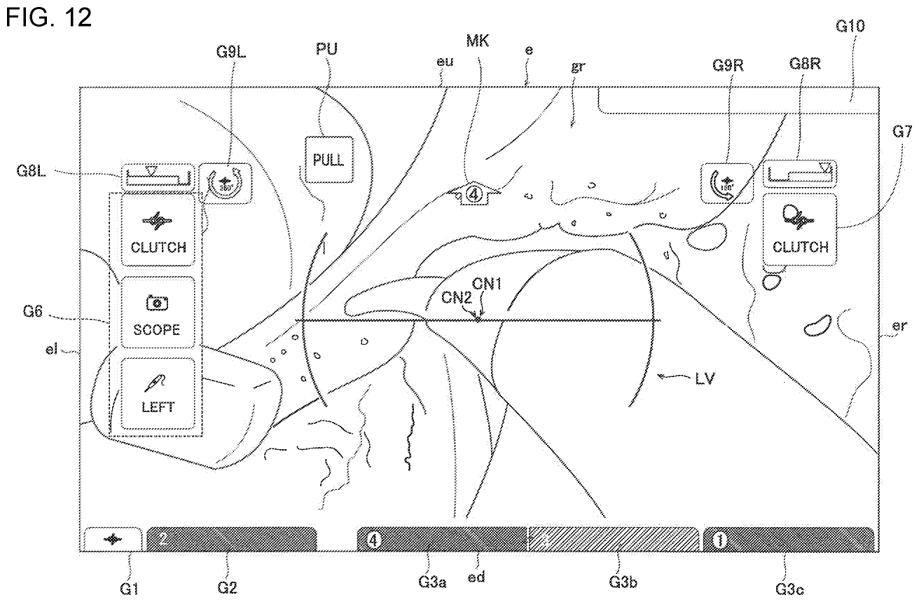

As illustrated in FIG. 1, the monitor 24 is a display device of a scope type configured to display an image (see FIG. 12) captured by the endoscope 6. The support arm 25 supports the monitor 24 in such a manner that the height of the monitor 24 is adjusted to the height of the face of the operator (such as a doctor). The touch panel 23 is disposed on the support bar 26. When a sensor(s) provided in the vicinity of the monitor 24 detects the head of the operator, the medical manipulator 1 is allowed to be operated by the remote control apparatus 2. The operator operates the operation handles 21 and the foot pedals 22, while viewing the surgical site (or affected area) displayed on the monitor 24. With this, the command (instruction) is inputted to the remote control apparatus 2. The instruction that is input to the remote control apparatus 2 is transmitted to the medical manipulator 1

The medical trolley 3 is provided with a control unit 31 (circuitry and/or processor) that controls the operation of the medical manipulator 1 and a storage 32 that stores therein programs for controlling the operation of the medical manipulator 1. The control unit 31 controls the operation of the medical instrument 4 according to commands (instructions) received through the operation handles 21, the foot pedals 22, or the arm operation unit 80. Specifically, based on the command inputted to the remote control apparatus 2, the control unit 31 of the medical trolley 3 controls the operation of the medical manipulator 1. Note that the control unit 31 is an example of an “control device” or a “controller.”

Further, the medical trolley 3 is provided with an input device 33. The input device 33 is configured to accept operations to move or change posture of a positioner 40, an arm base 50, and arms 60, mainly to prepare for surgery before the surgery. Further, the input device 33 is configured to receive input of information necessary to prepare the surgery. Note that the input device 33 is an example of an “surgical site input device” or an “user interface.”

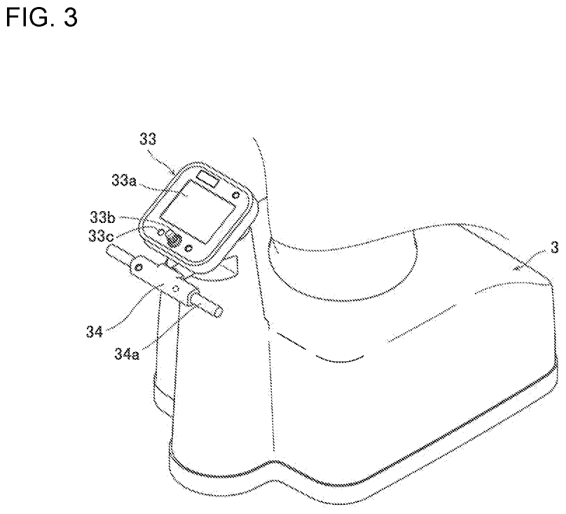

As illustrated in FIG. 3, the input device 33 of the medical trolley 3 is provided with a display 33a formed of a touch panel display and with a joystick 33b in the vicinity of the display 33a to control the movement of the positioner 40. By selecting one of the operation modes displayed on the display 33a and operating the joystick 33b, the positioner 40 is operated (moved) three-dimensionally. The enable switch 33c that enables or disables movement of the positioner 40 is provided in the vicinity of the joystick 33b. In a state where the movement of the positioner 40 is enabled by pressing the enable switch 33c, the positioner 40 is moved by operating the joystick 33b. In addition, on the touch panel of the display 33a, one of surgical sites (for example, anatomy: “prostate”, etc. see FIG. 15) and an insertion direction in which the medical manipulator 1 is to be inserted to the patient P (for example, “from the right side,” etc.) are selected.

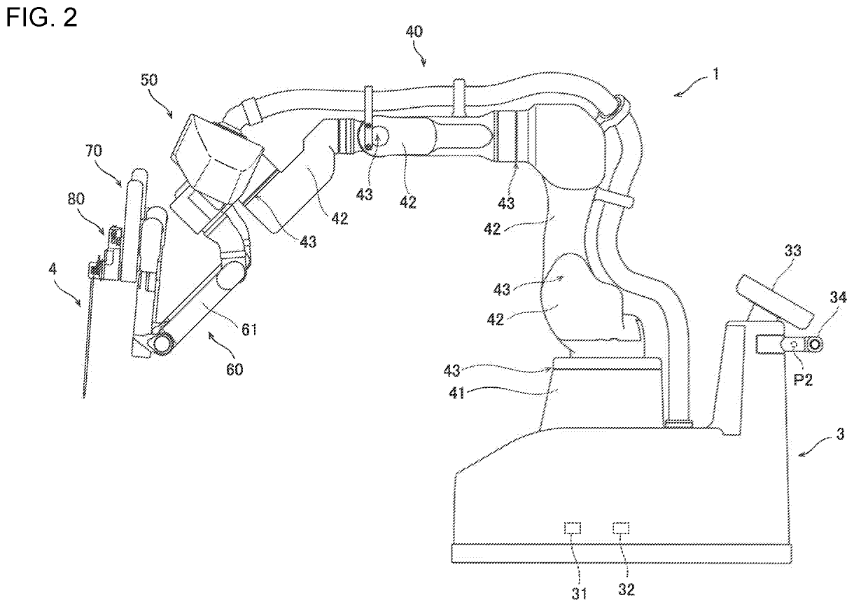

As illustrated in FIGS. 1 and 2, the medical manipulator 1 is disposed in the surgery room. The medical manipulator 1 includes the medical trolley 3, the positioner 40, the arm base 50, and the arms 60. The arm base 50 is attached to a distal end of the positioner 40. The arm base 50 is a relatively long rod shape (elongate shape). Base portions (proximal end portions) of the arms 60 are attached to the arm base 50. Each of the arms 60 is configured such that the arm 60 is able to take a folded posture (storage posture). The arm base 50 and the arms 60 are used with being covered with a sterile drape (not illustrated). The arm 60 supports the medical instrument 4.

The positioner 40 is configured as a 7-axis articulated robot. The positioner 40 is disposed on the medical trolley 3. The positioner 40 is configured to move the arm base 50. Specifically, the positioner 40 is configured to move the position of the arm base 50 three-dimensionally.

The positioner 40 includes a base portion 41 and link portions 42 connected to the base portion 41. The link portions 42 are connected to each other via joints 43.

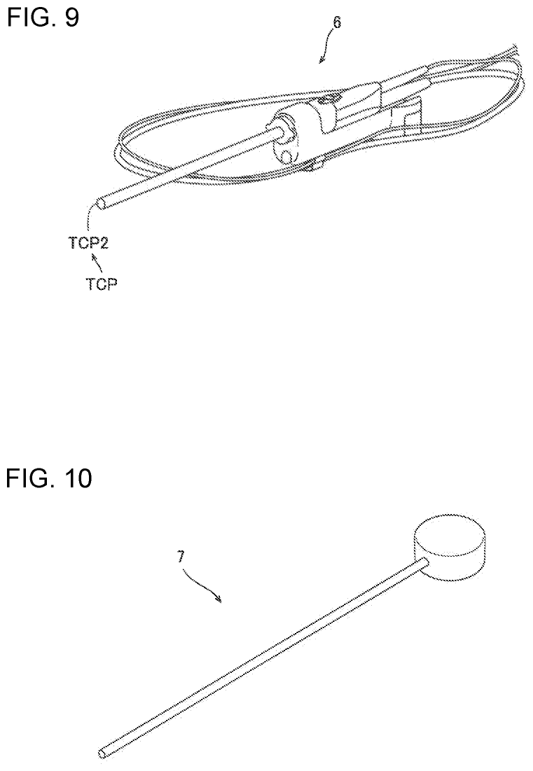

As illustrated in FIG. 1, to the distal end of each of the arms 60, the medical instrument 4 is attached. The medical instruments 4 include, for example, an instrument that is replaceable, an endoscope 6 (see FIG. 9) configured to capture an image of a surgical site, and the like.

The surgical operation system 100 is provided with a monitor cart 8 as illustrated in FIG. 1. The monitor cart 8 is provided with a display 8a. The display 8a is provided separately from the monitor 24 of the remote control apparatus 2. The monitor cart 8 is also provided with an image processing device 8b. The display 8a of the monitor cart 8 displays the image same as the image displayed in the monitor 24 of the remote control apparatus 2. That is, the image that is displayed on the monitor 24 and viewed by the surgeon through the monitor 24 can be also viewed by the worker (nurse, assistant, etc.) around the medical manipulator 1 and the patient P by means of the display 8a of the monitor cart 8. Note that the image processing device 8b is an example of an “control device” or a “controller.” Note that the display 8s is an example of a “display device.”

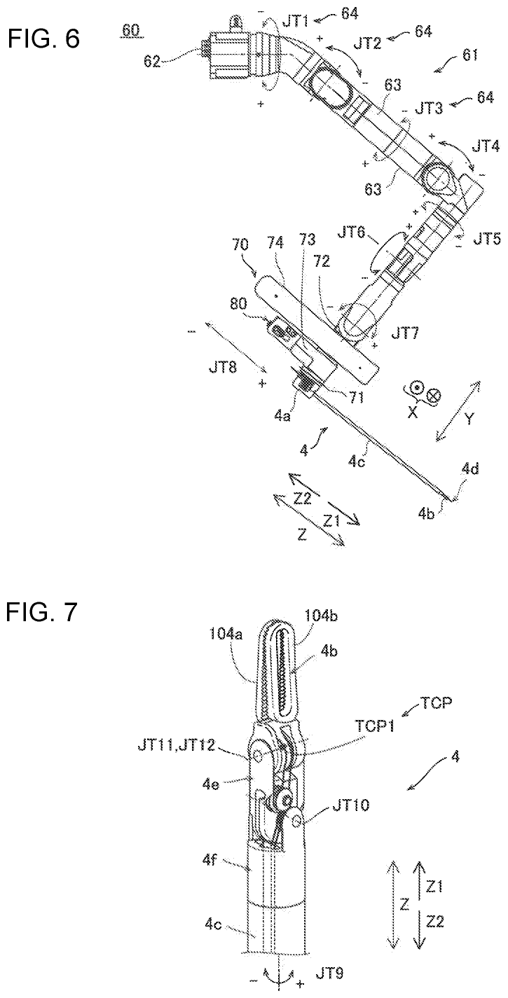

As illustrated in FIG. 6, the instrument is provided with a driven unit 4a, which is driven by servomotors M2 provided in a holder 71 of the arm 60. To the distal end of the instrument, forceps 4b as an end effector is provided.

As illustrated in FIG. 7, the instrument includes: a first support 4e having a distal end portion thereof that rotatably supports proximal end portions of jaw members 104a and 104b about an axis (joint) JT11; a second support 4f having a distal end portion thereof that rotatably supports a proximal end portion of the first support 4e about an axis (joint) JT10; and a shaft 4c connected a proximal end portion of the second support 4f. The jaw members 104a and 104b are configured to be opened and closed to each other. The driven unit 4a, the shaft 4c, the second support 4f, the first support 4e, and the forceps 4b are arranged along the Z direction. The axis JT11 is orthogonal to a direction (Z direction) in which the shaft 4c extends. The axis JT10 is provided away from the axis JT11 in the direction in which the shaft 4c extends, and is orthogonal to the axis JT11 and orthogonal to the direction in which the shaft 4c extends.

The forceps 4b is attached to the first support 4e so as to be rotatable about the axis JT11. The second support 4f rotatably supports the first support 4e about the axis JT10. In other words, the first support 4e is attached to the second support 4f so as to be rotatable about the axis JT10. A distal side (Z1 side) portion of the first support 4e has a U-shape. A tool center point (TCP1, Clevis) is set at the center of the U-shaped distal side portion of the first support 4e along the axis JT11.

The medical instrument 4 (forceps 4b) includes an axis (joint) JT9 as a rotation axis of the shaft 4c (an axis (joint) extending along the direction in which the shaft 4c extends) and an axis (joint) JT12 about which the forceps 4b open and close. Note that the number of the servomotors M2 are provided in the holder 71 of the arm 60 is two or more (for example, four). Rotors (rotation members) in the driven unit 4a are driven by the plurality of servomotors M2. As a result, the medical instrument 4 is driven about the J9, J10, J11, and J12 axes.

As illustrated in FIG. 9, a tool center point TCP2 of the endoscope 6 is set at the distal end of the endoscope 6.

Next, a configuration of the arm 60 is described in detail.

As illustrated in FIG. 6, the arm 60 includes an arm portion 61 (the base portion 62, the link portions 63, the joint portions 64) and a translational movement mechanism 70 provided at the distal end portion of the arm portion 61. The arm 60 is configured such that the distal end portion thereof is three-dimensionally movable with respect to the proximal side (the arm base 50) of the arm 60. The arm portion 61 is configured as a 7-axis articulated robot arm. The plural arms 60 have the same configuration.

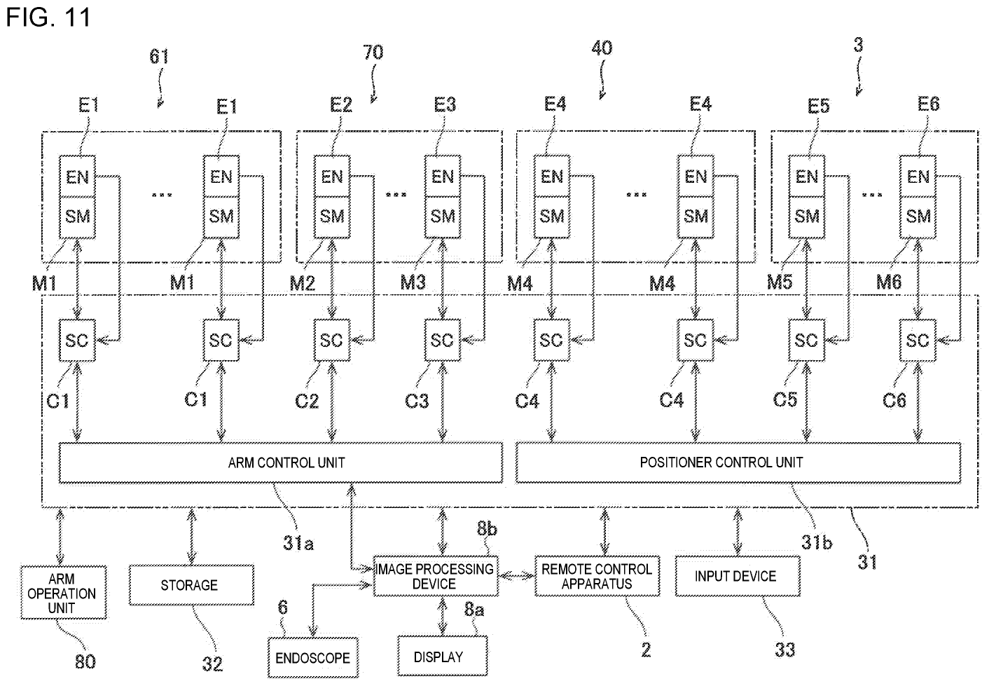

As illustrated in FIG. 6, the arm 60 includes the axis (joints) JT1 to JT7 as rotation axes and an axis (joint) J8 as a linear motion axis. The joints JT1 to JT7 correspond to the rotation axes of the joint portions 64 of the arm portion 61. The joint JT7 corresponds to the proximal end side link portion 72 of the translational movement mechanism 70. A joint JT8 is an axis for moving the distal end side link portion 73 of the translational movement mechanism 70 relative to the proximal end side link portion 72 along the Z direction. That is, the servomotors M1 illustrated in FIG. 11 are provided to correspond to the joints JT1 to JT7 of the arm 60. The servomotor M3 is provided to correspond to the joint JT8.

The translational movement mechanism 70 is provided on a side of the distal end of the arm portion 61. The medical instrument 4 is attached to the translational movement mechanism 70. The translational movement mechanism 70 translationally moves the medical instrument 4 in the insertion direction of the medical instrument 4 into a patient P. The translational movement mechanism 70 is configured to translationally move the medical instrument 4 relative to the arm portion 61. Specifically, the translational movement mechanism 70 is provided with the holder 71 configured to hold the medical instrument 4. The holder 71 accommodates therein the servo-motors M2 (see FIG. 11).

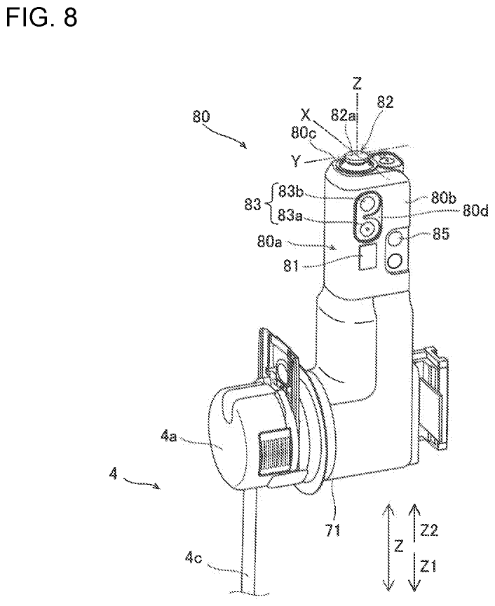

As illustrated in FIG. 8, the medical manipulator 1 includes an arm operation unit 80 (an arm manipulation unit 80) which is attached to each of the arms 60 to operate the arm 60. The arm operation unit 80 accepts commands (instructions) inputted by the operator O for operating the medical equipment 4. The arm operation unit 80 includes an enable switch 81, a joystick 82, and a switch section 83. The enable switch 81 enables or disables the joystick 82 and the switch section 83 to move the arm 60. When the enable switch 81 is depressed by an operator (nurse, assistant, etc.) gripping the arm operation unit 80, the movement of the medical instrument 4 by the arm 60 is enabled. Also, the joystick 82 and the switch section 83 are used to operate the arm 60. Note that the arm operation unit 80 is an example of an “operating device.”

The switch section 83 includes: a switch 83a for moving the medical instrument 4 in the direction in which the medical instrument 4 is inserted into the patient P along the longitudinal direction of the medical instrument 4; and a switch 83b for moving the distal end 4d of the medical instrument 4 in the direction opposite to the direction in which the medical instrument 4 is inserted into the patient P. Both the switch 83a and the switch 83b are composed of push button switches.

As illustrated in FIG. 8, the arm operation unit 80 includes a pivot button 85 for setting a pivot position PP that serves as a fulcrum for the movement of the medical instrument 4 attached to the arm 60. The pivot button 85 is provided on a surface 80b of the arm operation unit 80 so as to be adjacent to the enable switch 81. The pivot position PP is set by pressing the pivot button 85 in a state where the distal end of the endoscope 6 (see FIG. 9) or the distal end of the pivot position setting device 7 (FIG. 10) is moved to a position corresponding to an insertion position of the trocar T inserted into the body surface S of the patient P. The pivot position PP set is stored in the storage 32. Note that the pivot position PP is set as one point (coordinate) in the setting of the pivot position PP.

As illustrated in FIG. 1, the endoscope 6 is attached to one (for example, the arm 60c) of the plural arms 60, and the medical instruments 4 other than the endoscope 6 are attached to the other arms 60 (for example, the arms 60a, 60b, and 60d). Specifically, in surgery, the endoscope 6 is attached to one of the four arms 60, and the medical instruments 4 (forceps 4b, etc.) other than the endoscope 6 are attached to the other three arms 60. In the state where the endoscope 6 is attached to the arm 60, the pivot position PP for the endoscope 6 is set to the arm 60 to which the endoscope 6 is attached. Further, in the state where the pivot position setting device 7 is attached to the arm 60 to which the medical instrument 4 other than the endoscope 6 is attached, the pivot position PP for the medical instrument 4 is set to the arm 60 to which the medical instrument 4 other than the endoscope 6 is attached. The endoscope 6 is attached to one of two arms 60 (arms 60b and 60c) arranged in the center area among the four arms 60 arranged adjacent to each other. That is, the pivot position PP is individually set for each of the plurality of arms 60. The arm 60c is an example of a “first manipulator.” The arm 60a, 60b, or 60d is an example of a “second manipulator.”

As illustrated in FIG. 11, the arm 60 is provided with the plurality of servomotors M1, a plurality of encoders E1, and a plurality of speed reducers (not illustrated), so as to correspond to the plurality of joint portions 64 of the arm portion 61. The encoder E1 is configured to detect the rotation angle of the servomotor M1. The speed reducer is configured to reduce the rotation of the servomotor M1 to increase the torque.

As illustrated in FIG. 11, the translational movement mechanism 70 includes the servomotors M2 for rotating the rotors (rotation members) provided in the driven unit 4a of the medical instrument 4, a servomotor M3 for translationally moving the medical instrument 4, encoders E2, an encoder E3, and speed reducers (not illustrated). The encoders E2 and the encoder E3 are configured to detect the rotation angles of the servomotors M2 and the servomotor M3, respectively. The speed reducers are configured to reduce the rotations of the servomotors M2 and the servomotor M3 to increase the torque thereof.

The positioner 40 is provided with a plurality of servomotors M4, a plurality of encoders E4, and a plurality of speed reducers (not illustrated), so as to correspond to the plurality of joints 43 of the positioner 40. The encoders E4 detect the rotation angles of the servomotors M4. The speed reducers are configured to reduce the rotations of the servomotors M4 to increase the torque thereof.

The medical trolley 3 is provided with servomotors M5 that drive a plurality of front wheels (not illustrated) of the medical trolley 3 respectively, encoders E5, speed reducers (not illustrated), and brakes (not illustrated). The speed reducer is configured to reduce the rotation of the servomotor M5 to increase the torque. A throttle 34a of the medical trolley 3 is provided with a potentiometer P1 (see FIG. 1). The servomotors M5 for the front wheels are driven based on the rotation angle detected by the potentiometer P1 according to the rotation of the throttle portion 34a. The rear wheels (not illustrated) of the medical trolley 3 are a twin-wheel type and are steered based on the left-right rotation of an operation handle 34. The operation handle 34 of the medical trolley 3 is provided with a potentiometer P2 (see FIG. 2). The rear wheels of the medical trolley 3 are provided with servomotors M6, encoders E6, and speed reducers (not illustrated). The speed reducer is configured to reduce the rotation of the servomotor M6 to increase the torque. The servomotors M6 for the rear wheels are driven based on the rotation angle detected by the potentiometer P2 according to the left-right rotation of the operation handle 34. That is, the steering of the rear wheels by the left-right rotation of the operation handle 34 is power-assisted by the servomotors M6.

Further, the medical trolley 3 moves in the front-rear direction by driving the front wheels. By rotating the operation handle 34 of the medical trolley 3, the rear wheels of the medical trolley 3 are steered and thus the medical trolley 3 is rotated in the left-right direction.

The control unit 31 of the medical trolley 3 includes an arm control unit 31a that controls the movement of the plurality of arms 60 based on commands, and a positioner control unit 31b that controls the movement of the positioner 40 and driving of the front wheel (not illustrated) of the medical trolley 3 based on commands. A servo control unit C1 that controls the servomotors M1 for driving the arm 60 is electrically connected to the arm control unit 31a. Further, an encoder E1 that detects the rotation angle of the servomotor M1 is electrically connected to the servo control unit C1.

A servo control unit C2 that controls the servomotors M2 for driving the medical instrument 4 is electrically connected to the arm control unit 31a. The encoders E2 that detect the rotation angles of the servomotors M2 are electrically connected to the servo control unit C2. The servo control unit C3 that controls the servomotor M3 for translationally moving by the translational movement mechanism 70 is electrically connected to the arm control unit 31a. The encoder E3 for detecting the rotation angle of the servomotor M3 is electrically connected to the servo control unit C3.

The operation command inputted to the remote control apparatus 2 is inputted to the arm control unit 31a. The arm control unit 31a generates position commands based on the operation command inputted and the rotation angles detected by the encoders E1 (E2, E3), and outputs the position commands to the servo control units C1 (C2, C3). The servo control units C1 (C2, C3) generate torque commands based on the position commands inputted from the arm control unit 31a and the rotation angles detected by the encoders E1 (E2, E3), and output the torque commands to the servomotors M1 (M2, M3). As a result, the arm 60 is moved so as to comply with the operation command inputted to the remote control apparatus 2. The control unit 31 is configured to control the arm 60c to move the endoscope 6 about the pivot position PP. The control unit 31 is configured to control the arm 60a (60b, 60d) to move the medical instrument 4 about the pivot position PP.

As illustrated in FIG. 11, the control unit 31 (arm control unit 31a) is configured to operate the arm 60 based on an input signal from the joystick 82 of the arm operation unit 80. Specifically, the arm control unit 31a generates position commands based on the input signal (operation command) inputted from the joystick 82 and the rotation angles detected by the encoders E1, and outputs the position commands to the servo control units C1. The servo control unit C1 generates torque commands based on the position command inputted from the arm control unit 31a and the rotation angles detected by the encoders E1, and outputs the torque commands to the servomotors M1. As a result, the arm 60 is moved so as to follow the operation command inputted to the joystick 82.

The control unit 31 (arm control unit 31a) is configured to operate the arm 60 based on an input signal from the switch section 83 of the arm operation unit 80. Specifically, the arm control unit 31a generates position commands based on the input signal (operation command) inputted from the switch section 83 and the rotation angles detected by the encoders E1 or E3, and outputs the position commands to the servo control units C1 or C3. The servo control units C1 or C3 generate torque commands based on the position command inputted from the arm control unit 31a and the rotation angles detected by the encoders E1 or E3, and outputs the generated torque commands to the servomotors M1 or M3. As a result, the arm 60 is moved so as to follow the operation command inputted to the switch section 83.

As illustrated in FIG. 11, the servo control units C4 that control the servomotors M4 for moving the positioner 40 are electrically connected to the positioner control unit 31b. The encoders E4 that detects the rotation angles of the servomotors M4 are electrically connected to the servo control units C4. The servo control units C5 that control the servomotors 5 for driving the front wheel (not illustrated) of the medical trolley 3 are electrically connected to the positioner control unit 31b. The encoders E5 that detect the rotation angles of the servomotors M5 are electrically connected to the servo control units C5.

An operation command regarding setting of the preparation position and the like is inputted from the input device 33 to the positioner control unit 31b. The positioner control unit 31b generates position commands based on the operation command inputted from the input device 33 and the rotation angle detected by the encoder E4, and outputs the position commands to the servo control units C4. The servo control unit C4 generates torque commands based on the position command inputted from the positioner control unit 31b and the rotation angles detected by the encoders E4, and outputs the torque commands to the servomotors M4. As a result, the positioner 40 is moved so as to follow the operation command inputted to the input device 33. Similarly, the positioner control unit 31b moves the medical trolley 3 based on the operation command from the input device 33.

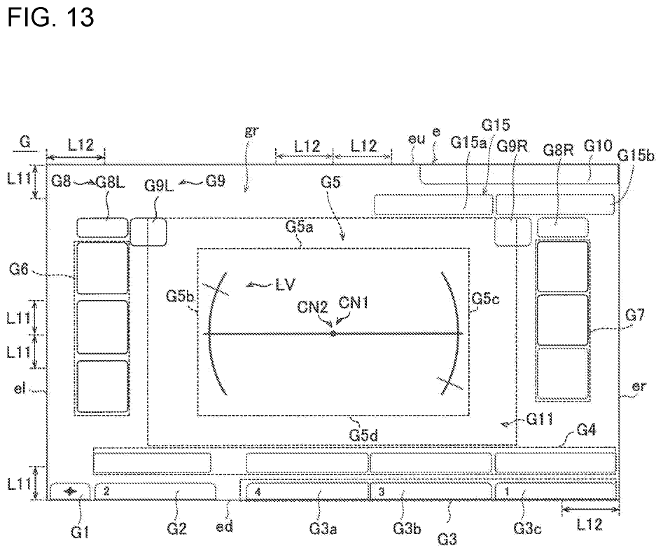

The surgical operation system 100 includes the image processing device 8b. The image processing device 8b is configured to execute processing based on predetermined programs. The image processing device 8b is configured by a computer. The image processing device 8b includes a processor or a circuitry such as a CPU that executes programs, and a storage such as a memory that stores the programs. The image processing device 8b is configured to generate a graphical user interface G (see FIG. 13) and display, on the monitor 24 of the remote control apparatus 2, the graphical user interface G superimposed on the endoscope image (see FIG. 12) captured by the endoscope 6. The image processing device 8b is also configured to display, on the display 8a, the graphical user interface G superimposed on the endoscope image captured by the endoscope 6. The image processing device 8b is configured to obtain the image from the endoscope 6. The image processing device 8b is configured to be able to communicate with the control unit 31.

As illustrated in FIG. 13, the graphical user interface G includes a clutch area G1. A state of the clutch pedal 22b is displayed in the clutch area G1.

As illustrated in FIG. 13, in an embodiment, the graphical user interface G includes a camera area G2 indicating information relating to the endoscope 6. The camera area G2 is displayed in an area near the lower end ed (see FIG. 12) of a screen gr of the monitor 24.

As illustrated in FIG. 13, the graphical user interface G includes hand areas G3. The hand areas G3 display information of the medical instruments 4 attached to the arms 60 and indicates operation states of the medical instruments 4 attached to the arms 60 (and thus may serve as medical instrument operation state indicators). The hand areas G3 include: a hand area G3a that displays information about the arm 60a (the arm 60a having the identification number “4”) and the medical instrument 4 attached to the arm 60a that are operated by the operation handle 21L for the left hand, a hand area G3b that displays information about the arm 60b (the arm 60b having the identification number “3”) and the medical instrument 4 for replacement that is attached to the arm 60b, and a hand area G3c that displays information about the arm 60d (the arm 60d having the identification number “1”) and the medical instrument 4 that is attached to the arm 60d that are operated by the operation handle 21R for the right hand. The hand areas G3 are displayed in an area in the vicinity of the lower end ed of the screen gr of the monitor 24. The clutch area G1 is also displayed in the area in the vicinity of the lower end ed of the screen gr of the monitor 24. Specifically, as illustrated in FIG. 13, the clutch area G1, the camera area G2, and the hand area G3 are displayed between the lower end ed of the screen gr and a position above the lower end ed of the screen gr by a length (L11) of one tenth of the vertical length of the screen gr.

The information about the arm 60 include: the identification number (e.g., “1”, “2”, etc.) of the arm 60; and an arrow icon that is displayed when the arm 60 is set as a replacement destination of an arm 60 to which the medical instrument 4 for replacement is attached. The information about the medical instrument 4 include a name (a type) of the medical instrument 4. The information about the states of the coagulation pedal 22e and the cutting pedal 22d include the operation state of the clutch pedal 22b, the operation state of the coagulation pedal 22e, and the operation state of the cutting pedal 22d.

Further, as illustrated in FIG. 12, the hand areas (hand area G3a and hand area G3c) for the arms 60 that are operation targets to be operated are displayed in dark gray. In these dark gray hand areas G3a and G3c, the identification numbers “1” and “4” of the arms 60 that are the operation targets are displayed in white ovals (white elongate circles). Further, the hand area (hand area G3b) of the arm 60 that is not the operation target is displayed in light gray, and in this hand area G3b, the identification number “3” of that arm 60 is displayed in an even lighter gray color lighter than the light gray color of the hand area G3b.

As illustrated in FIG. 13, the graphical user interface G includes a medical instrument usage information area G4, which is a pop-up area. In the medical instrument usage information area G4, the current number of times of use/the maximum number of times of use of the medical instrument 4 attached to each arm 60 is displayed in a pop-up. When the current number of uses of the medical instrument becomes equal to the maximum number of uses of the medical instrument, the current number of uses is displayed in red. When an error occurs in the medical instrument 4 attached to any one of the arms 60, error information is displayed in a pop-up manner. When the medical instrument 4 is not attached to the arm 60, nothing is displayed in the medical instrument usage information area G4. The medical instrument usage information area G4 is displayed in an area adjacent above the clutch area G1, the camera area G2, and the hand area G3 on the monitor 24.

As illustrated in FIG. 13, the graphical user interface G includes a level indication area G5. In the level indication area G5, information on the angle of the endoscope 6 is displayed. The level indication area G5 is displayed only while the camera pedal 22c is being depressed (stepped). That is, when receiving the command that enables the movement of the endoscope 6, the image processing device 8b displays a level LV (a level indication LV) of the endoscope 6 in the level indication area G5.

As illustrated in FIG. 13, the graphical user interface G includes a left pop-up area G6. The left pop-up area G6 displays, in the hover state which is a state where the foot is placed on one of the foot pedals 22, an icon that indicates the one of the pedals 22 on which the foot is placed. The left pop-up area G6 is displayed in a left side portion on the monitor 24.

As illustrated in FIG. 13, the graphical user interface G includes a right pop-up area G7. In the right pop-up area G7, an icon is displayed when the foot is placed on the coagulation pedal 22eR or the cutting pedal 22dR. The right pop-up area G7 is displayed in a right side portion on the monitor 24.

Further, as illustrated in FIG. 13, the graphical user interface G includes the first area G8 that displays the movable range of the arm 60 and the operable range of the operation handle 21 where the operation handle 21 is operable in the movable range of the arm 60. The graphical user interface G also includes a second area G9 that displays a direction in which the operation handle 21 is required to be operated to return the operation handle 21 to the inside of the operable range of the operation handle (toward the center of the operable range of the operation handle) and/or (e.g., “and” in an embodiment) to return the arm 60 to the inside of the movable range (toward the center of the movable range of the arm 60).

Further, the number of arms 60 that can be operated by the operation handle 21 is two. For example, the operation handle 21L operates the left arm 60L (for example, the arm 60a, see FIG. 1) that supports the medical instrument 4. Further, the operation handle 21R operates the right arm 60R (for example, the arm 60d, see FIG. 1) that supports the medical instrument 4. The first area G8 includes a first area G8L for the left arm 60L and a first area G8R for the right arm 60R, and the second area G9 includes a second area G9L for the left arm 60L and a second area G9R for the right arm 60R.

As illustrated in FIG. 13, the graphical user interface G includes error notification areas G15 (G15a, G15b) The error notification area G15a is displayed in a pop-up to display warning and error information when a warning or an error occurs. The error notification area G15b is displayed in a pop-up to display details of notes of the warning and the error displayed in the error notification area G15a.

As illustrated in FIG. 13, the graphical user interface G includes a status area G10. In the status area G10, information such as the remaining amount of the built-in battery of the medical manipulator 1, the brightness/contrast of the monitor 24, the lap time, and the elapsed time of the surgery are displayed. Specifically, as illustrated in FIG. 13, the status area G10 is displayed between the upper end eu of the screen gr and a position below the upper end eu of the screen gr by a length (L11) of one tenth of the vertical length of the screen gr.

The control unit 31 is configured to determine, based on the position of the distal end of the medical instrument 4 and the field of view of the endoscope 6, whether or not the distal end of the medical instrument 4 is located outside the field of view of the endoscope 6. Specifically, the control unit 31 is configured to determine whether or not each of the medical instruments is positioned outside the field of view of the endoscope 6, based on imaging range information of the endoscope 6 and the position information of the distal ends of the medical instruments 4 supported by the arms 60. That is, the control unit 31 acquires the position of each medical instrument 4 based on the posture and the position of the arm 60 to which the medical instrument is attached. Further, the control unit 31 acquires the imaging direction of the endoscope 6 based on the posture and the position of the arm 60. Further, the control unit 31 acquires the field angle (field of view range) of the endoscope 6 based on the zoom state of the endoscope 6. For example, the control unit 31 obtains the field angle (field of view range) of the endoscope 6 with reference to values of the mechanical system (lens, etc.) of the endoscope 6. Then, the control unit 31 obtains the coordinates of the distal end of each of the medical instruments 4 with respect to the field of view of the endoscope 6, based on the information on the field of view of the endoscope 6, the posture and the position of the endoscope 6, and the positions of the arms 60. With this, the control unit 31 determines whether each of the medical instruments 4 is located outside the field of view of the endoscope 6.

Further, when the distal end position of the medical instrument 4 is located outside the field of view of the endoscope 6, the control unit 31 is configured to change the operation of the medical instrument 4 in response to a command received by the operation handle 21, the foot pedal 22 (the cutting pedal 22d or the coagulation pedal 22e), or the arm operation unit 80, from a first operation mode (an normal operation mode) of the surgical instrument for the state where the distal end of the medical instrument 4 is located in the field of view of the endoscope 6 to a second operation mode different from the first operation mode.

Specifically, the command received by the operation handle 21 or the arm operation unit 80 includes a movement command to move the medical instrument 4. The command received by the cutting pedal 22d or the coagulation pedal 22e includes an energization command to supply electrical energy to the medical instrument 4. The command received by the pair of grip members 21f of the operation handle 21 includes an opening/closing command to open or close the pair of the jaws provided at the distal end of the medical instrument 4.

Further, when the distal end position of the medical instrument 4 changes from being outside the field of view of the endoscope 6 to being within the field of view, the control unit 31 releases the second operation mode that changes the operation of the medical instrument 4 that corresponds to the command received by the operation handles 21, the foot pedals 22, or the arm operation unit 80. That is, the control unit 31 restricts the operation of the medical instrument 4 when the distal end position of the medical instrument 4 comes out of the field of view of the endoscope 6 due to movement of the medical instrument 4 and/or due to change of the field of view of the endoscope 6. To the contrary, the control unit 31 releases the restriction on the operation of the medical instrument 4 when it is determined that the distal end position of the medical instrument 4 comes in the field of view of the endoscope 6 due to movement of the endoscope 6 and/or due to change of the field of view of the endoscope 6. Specifically, by operating both the operation handle 21R and the operation handle 21L while the camera pedal 22c is depressed by the operator, the distal end position of the medical instrument 4 outside the field of view is brought into the field of view of the endoscope 6.

As an example of an embodiment, the control unit 31 is configured, when the distal end position of the medical instrument 4 is located outside the field of view of the endoscope 6, to control the arms 60a, 60b, and 60c not to move the medical instrument 4 in response to (regardless of) the movement command to move the medical instrument 4. That is, the control unit 31 is configured, when the distal end position of the medical instrument 4 comes out of the field of view of the endoscope 6 due to the movement of the medical instrument 4 or the like, not to move the medical instrument 4 that is located outside of the field of view of the endoscope 6 even if the operating handle 21 is operated to move the medical instrument 4 that is located outside the field of view.

As another example of an embodiment, the control unit 31 is configured, when the distal end position of the medical instrument 4 is located outside the field of view of the endoscope 6, to control the arms 60a, 60b and 60d to move, in response to the movement command, the medical instrument 4 with scaling smaller than a predetermined scaling for the normal operation mode (the state where the medical instrument is in the field of view of the endoscope 6). For example, when the predetermined scaling for the normal operation mode (the state where the medical instrument is in the field of view of the endoscope 6) is set to 1/m times, the scaling for moving the medical instrument 4 that is located outside the field of view of the endoscope is set to 1/n times (where m<n).

As another example of an embodiment, the control unit 31 is configured, when the distal end position of the medical instrument 4 is located outside the field of view of the endoscope 6, to control the energization of the medical instrument 4 so as to prevent the supply of electrical energy in response to (regardless of) the energization command. For example, even when receiving the energization command for incision or coagulation to supply the electrical energy to the medical instrument 4 that is located outside the field of view of the endoscope 6, the control unit 31 does not energize the medical instrument 4 that is located outside the field of view.

As another example of an embodiment, the control unit 31 is configured, when the distal end position of the medical instrument 4 is located outside the field of view of the endoscope 6, to control the motion of the jaws not to open or close the jaws of the medical instrument 4 in response to (regardless of) the opening/closing command.

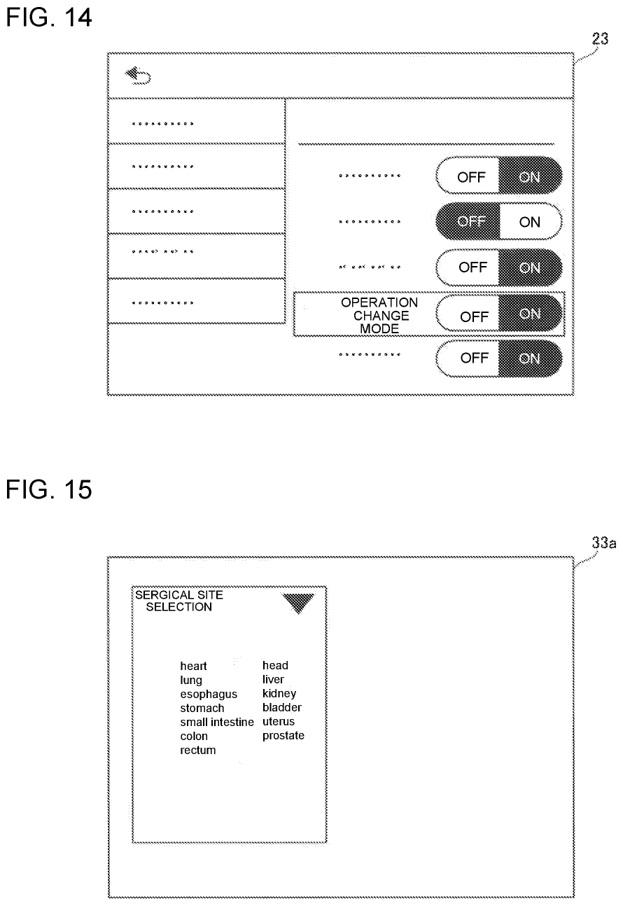

As illustrated in FIG. 14, the touch panel 23 is used to set (turn on) an operation change mode. As another example of an embodiment, in a state where the operation change mode is turned on, the movement of the medical instrument 4 that is located outside the field of view of the endoscope 6 is restricted. Further, in a state where the operation change mode is turned off, even if the medical instrument 4 is located outside the field of view of the endoscope 6, the operation of the medical instrument 4 outside of the field of view of the endoscope 6 is not restricted.

That is, in the state where the operation change mode is set (is turned on) though the touch panel 23, the control unit 31 changes (modifies), when it is determined that the distal end position of the medical instrument 4 is located outside the field of view of the endoscope 6, the operation of the medical instrument 4 that corresponds to the command. To the contrary, in a state where the operation change mode is not set (is turned off) through the touch panel 23, the control unit 31 controls the operation of the medical instrument 4 according to the command without changing (modifying) the operation of the medical instrument 4 that corresponds to the command, even when the distal end position of the medical instrument 4 is outside the field of view of the endoscope 6.

As illustrated in FIG. 15, the input device 33 is used to receive input (selection) of one of surgical sites. Then, the medical manipulator 1 is controlled to operate appropriately according to the selected surgical site.

As another example of an embodiment, in a state where one of first surgical sites is set (selected) through the input device 33, the control unit 31 changes (modifies), when it is determined that the distal end position of the medical instrument 4 is outside the field of view of the endoscope 6, the operation of the medical instrument 4 that corresponds to the command. For example, the first surgical sites include parts of body, such as lung or the like, where blood vessels such as the aorta and the vena cava are nearby. When one of the first surgical sites is selected, the movement of the medical instrument 4 that is located outside the field of view of the endoscope 6 is restricted. It may be preferable that such a restriction on the movement of the medical instrument 4 that is located outside the field of view of the endoscope 6 is to prohibit the movement of the medical instrument 4 that is located outside of the field of view of the endoscope 6 in response to (regardless of) the movement command to move the medical instrument 4. It should be noted that the touch panel 23 can be used to set the surgical site(s) that is to be restricted and the type of operation restriction.

In a state where one of second surgical sites which are different from the first surgical sites is set (selected) through the input device 33, the control unit 31 does not change (modify), even when the distal end position of the medical instrument 4 is outside the field of view of the endoscope 6, the operation of the medical instrument 4 that corresponds to the command and thus operates the medical instrument 4 according to the command. For example, the second surgical sites include parts of body, such as a prostate or the like, where blood vessels such as the aorta and the vena cava are not nearby. Then, in the state where one of the second surgical sites is selected, even when the medical instrument 4 is located out of the field of view of the endoscope 6, the operation of the medical instrument 4 that is located outside the field of view of the endoscope 6 is not restricted.

Further, the image processing device 8b obtains from the control unit 31 information on the determination result as to whether or not the medical instrument 4 is located outside the field of view of the endoscope 6, and generates the graphical user interface G based on the obtained information. When the distal end of the medical instrument 4 is located outside the field of view of the endoscope 6, the image processing device 8b displays a mark MK indicating that the medical instrument 4 is located outside the field of view of the endoscope 6 on the display 8a and the monitor 24. Note that the mark MK is an example of a “first indicator” or a first sign.”

As illustrated in FIG. 12, when it is determined that the medical instrument 4 that is being operated with the operation handle(s) 21 is located outside the field of view of the endoscope, the image processing device 8b displays the mark MK indicating the medical instrument 4 that is located outside the field of view of the endoscope in an inner area of the graphical user interface G which does not include the vicinity of the peripheral edge of the display screen. That is, the image processing device 8b acquires from the control unit 31 information that the medical instrument 4 being operated by the operation handle(s) 21 is located outside the field of view of the endoscope 6, and generates and displays the graphical user interface G including the mark MK corresponding to the medical instrument 4 that is located outside the field of view. As illustrated in FIG. 13, the image processing device 8b is configured to display the mark MK in a neighborhood area G11 that is provided in the vicinity of the peripheral edge (the outer edge) of the level indication area G5 of the graphical user interface G. That is, the mark MK (the graphical user interface G) is displayed in the neighborhood area G11, wherein the neighborhood area G11 is provided in the vicinity of the peripheral edge of the level indication area G5 including the center CN1 of the monitor screen gr and does not include the vicinity of the peripheral edge e (eu, ed, el, and er) of the screen gr of the monitor 24. Note that the neighborhood area G11 is located outside a level indication LV and in the vicinity of and adjacent to the level indication LV.

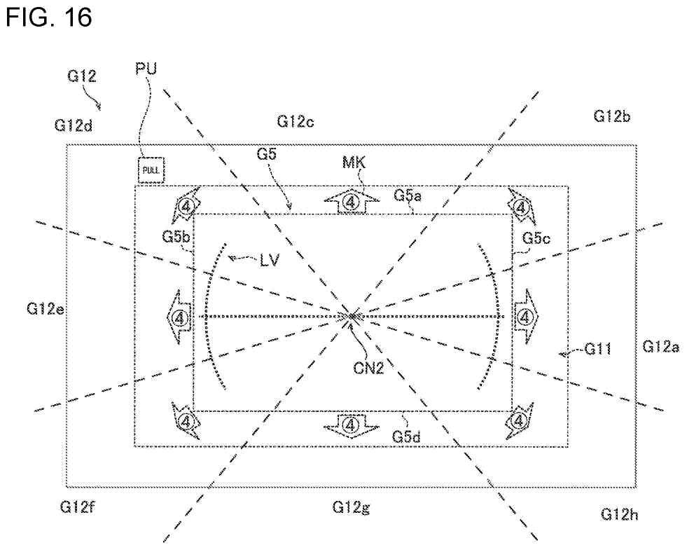

As illustrated in FIGS. 12 and 16, the mark MK includes an arrow that indicates a direction in which the medical instrument 4 that is located outside the field of view of the endoscope 6 is deviated from the field of view of the endoscope 6. Specifically, when it is determined that the medical instrument 4 that is being operated by the operation handle 21 for the left hand is located outside the field of view of the endoscope 6, the identification number of the medical instrument 4 that is located outside the field of view (for example, the number “4” displayed in the hand area G3, which is the information on the arm 60a) is displayed in the arrow. Note that the identification number may be displayed outside the arrow. The inside of the arrow is displayed as transparent or translucent so that the image captured by the endoscope can be seen through the arrow. When the medical instrument 4 is an operation target, the identification number in the mark MK corresponding to the target medical instrument 4 (the target arm 60) is displayed in black color in a white circle. When the medical instrument 4 is not an operation target, the identification number in the mark MK corresponding to the medical instrument 4 (the arm 60) is displayed in gray color in a black circle.

In addition, as illustrated in FIG. 16, the image processing device 8b is configured to display the mark MK that indicates which direction the medical instrument 4 is deviated from the field of view of the endoscope, e.g., the image processing device 8b is configured to display the mark MK that indicates which region the medical instrument 4 is located among eight regions into which an out-of-field area (an area outside of the field of view of the endoscope) is divided according to the shape of the rectangular display whose vertical and horizontal lengths are not equal.

Further, as illustrated in FIG. 16, the horizontal length of the display is greater than the vertical length of the display. The eight divided regions G12a, G12b, G12c, G12d, G12e, G12f, G12g, and G12h are provided such that angles 61 of the upper region G12c and the lower region G12g are larger than angles 62 and 63 of the right region G12a, the left region G12e, and the upper right region G12e, the upper left region G12d, the lower right region G12h, and the lower left region G12f.

Further, the image processing device 8b is configured such that each of the eight divided regions of the neighborhood area G11 includes a predetermined position to display the mark MK.

Further, when the distal end position of the medical instrument 4 is outside the field of view of the endoscope 6, the image processing device 8b displays a pull-back indicator PU that prompts to pull back the endoscope toward the proximal end side of the endoscope 6 (or a zoom-out indicator that prompts to zoom out) so as to cause the distal end position of the medical instrument 4 to come in the field of view of the endoscope 6. That is, the pull-back indicator PU indicates that if the endoscope 6 is pulled back toward the proximal end side of the endoscope 6 (zoom out), the distal end position of the medical instrument 4 will come in the field of view of the endoscope 6. Note that the pull-back indicator PU is an example of a “second indicator” or a “second sign.”

As illustrated in FIG. 16, the image processing device 8b is configured to display, the pull-back indicator PU in an area in the vicinity of and adjacent to the neighborhood area G11 of the level indication area G5 of the graphical user interface G. For example, the image processing device 8b displays the pull-back indicator PU at a position above the neighborhood area G11. Further, the image processing device 8b displays the pull-back indicator PU in a region outside and adjacent to the neighborhood area G11 in the left-right direction.

Also, as illustrated in FIG. 13, when receiving through the control unit 31 the command that enables the movement of the endoscope 6, the image processing device 8b displays the level indication LV of the endoscope 6 in the level indication area G5. That is, the image processing device 8b displays the level indicator of the endoscope 6 in the level indicator display area G5 of the graphical user interface G while the camera movement operation is being performed on the camera pedal 22c (while the camera pedal 22c is being operated to move the endoscope 6). The image processing device 8b displays, on the monitor 24, the graphical user interface G to display the mark MK in the neighborhood area G11 outside and in the vicinity of the peripheral edge (the outer edge) of the level indication area G5. The level indication LV indicates the inclination of the field of view of the endoscope 6 with respect to the patient P.

As illustrated in FIG. 13, the level indication area G5 is a rectangular area (a rectangular shape elongated in the lateral direction) including the upper side G5a, the left side G5b, the right side G5c, and the lower side G5d thereof. In this way, the visibility of the operator can be improved by displaying the mark MK in the neighborhood area G11 in the vicinity of the level indication area G5 having a predetermined size including the center of the screen gr. The upper side G5a of the level indication area G5 is provided between a position upper from the center CN1 of the screen gr of the monitor 24 by the length L11 of one tenth ( 1/10) of the vertical length of the screen gr and a position lower from the upper end eu of the screen gr by the length L11 of one tenth of the vertical length of the screen gr. It may be preferable that the upper side G5a of the level indication area G5 is provided between a position upper from the center CN1 of the screen gr of the monitor 24 by a length of one eighth of the vertical length of the screen gr and a position lower from the upper edge eu of the screen by the length of one eighth of the vertical length of the screen gr. It may be more preferable that the upper side G5a of the level indication area G5 is provided between a position upper from the center CN1 of the screen gr by a length of one sixth of the vertical length of the screen gr and a position lower from the upper edge eu of the screen by the length of one sixth of the vertical length of the screen gr.

The upper side G5a of the level indication area G5 is provided at a position lower than the status area G10 that displays the remaining battery level of the built-in battery of the medical manipulator 1, or the like.

The lower end G5d of the level indication area G5 is provided above the hand area G3a, the hand area G3b, the hand area G3c, and the camera area G2. Further, the lower end G5d of the level indication area G5 is provided above the medical instrument usage information area G4.

Note that the center CN1 of the screen gr of the monitor 24 and the center CN2 of the level indication area G5 are provided at positions substantially same as each other.

Further, the mark MK indicating the medical instrument 4 that is located outside the field of view is displayed between the level indication area G5 and at least one (e.g., “all” in an embodiment) of the medical instrument usage information area G4, the left pop-up area G6, the right pop-up area G7, and the status area G10. Note that the medical instrument usage information area G4 indicates the usage information of each medical instrument 4. The left pop-up area G6 is displayed when the foot pedal 22 is operated (that is, in the hover state where the foot is placed on the foot pedal 22). The right pop-up area G7 is displayed when the coagulation pedal 22eR or the cutting pedal 22dR is operated (that is, when the foot is placed on the coagulation pedal 22eR or the cutting pedal 22dR. The status area G10 displays the status of the surgical operation system 100.

As illustrated in FIG. 12, the mark MK includes the identification number to identify the arm 60 that supports the medical instrument 4 that is located outside the field of view of the endoscope 6. In an example illustrated in FIG. 12, the mark MK including the letter of the number “4” is displayed that indicates that the medical instrument 4 attached to the arm 60a (corresponding to the hand area G3 having the identification number “4”) is located outside the field of view of the endoscope 6.

### Effects of One or More Embodiments

According to one or more embodiments, effects as described below can be obtained.

In an embodiment described above, the control unit 31 is configured to determine, based on the relationship between the field of view of the endoscope 6 and the position of the distal end of the medical instrument 4, whether or not the distal end of the medical instrument 4 is located outside the field of view of the endoscope, and configured to change, when it is determined that the distal end of the medical instrument 4 is located outside the field of view of the endoscope, the operation of the medical instrument 4 that corresponds to the command received through the operation handles 21, the foot pedals 22, or the arm operation unit 80 from the first operation mode (the normal operation mode) for the state where the distal end of the medical instrument 4 is located in the field of view of the endoscope. Accordingly, when the distal end of the medical instrument 4 is located outside the field of view of the endoscope 6 and thus the operator cannot visually confirm the distal end of the medical instrument 4, the operation of the medical instrument 4 can be restricted such as not to operate the medical instrument 4. Therefore, it is possible to suppress the medical instrument 4 from unintentionally coming in contact with an object such as a part within a patient's body P.

Further in an embodiment described above, when the distal end position of the medical instrument 4 changes from being outside the field of view of the endoscope 6 to being within the field of view of the endoscope 6, the control unit 31 releases the second operation mode that changes the operation of the medical instrument 4 that corresponds to the command received by the operation handles 21, the foot pedals 22, or the arm operation unit 80. Accordingly, when the distal end position of the medical instrument 4 comes in the field of view of the endoscope 6, the operation of the medical instrument 4 is returned to the normal operation mode (the first operation mode), and thus the operator can operate the medical instrument 4 while visually confirming the distal end of the medical instrument 4.

Further in an embodiment described above, the control unit 31 is configured, when it is determined that the distal end position of the medical instrument 4 is located outside the field of view of the endoscope 6, to control the arms 60a, 60b, and 60c not to move the medical instrument 4 in response to (regardless of) the movement command that moves the medical instrument 4. Accordingly, since the medical instrument 4 that is located outside of the field of view of the endoscope 6 does not move, it is possible to effectively suppress the medical instrument 4 from unintentionally coming in contact with an object such as a part in the patient's body or the like.

Further in an embodiment described above, the control unit 31 is configured to move the medical instrument 4 according to the movement command with the predetermined scaling. Further, the control unit 31 is configured, when it is determined that the distal end position of the medical instrument 4 is located outside the field of view of the endoscope 6, to control the arms 60a, 60b and 60d to move the medical instrument 4 according to the movement command with scaling smaller than the predetermined scaling. Accordingly, since the medical instrument 4 that is located outside of the field of view of the endoscope 6 moves slower than in the state where the medical instrument 4 is located inside the field of view of the endoscope 6. Therefore, it is possible to suppress the medical instrument 4 from unintentionally coming in contact with an object such as a part in the patient's body or the like.

Further in an embodiment described above, the control unit 31 is configured, when the distal end position of the medical instrument 4 is located outside the field of view of the endoscope 6, to control the energization of the medical instrument 4 so as to prevent the supply of the electrical energy in response to (regardless of) the energization command. Accordingly, this prevents the energization of the medical instrument 4 that is located outside of the field of view of the endoscope 6 so as to suppress from unintentionally treating a part of the patient's body that is located outside the field of view of the endoscope 6.

Further in an embodiment described above, the control unit 31 is configured, when the distal end position of the medical instrument 4 is located outside the field of view of the endoscope 6, to control the motion of the jaws not to open or close the jaws of the medical instrument 4 in response to (regardless of) the opening/closing command. Accordingly, since the jaws of the medical instrument 4 that is located outside the field of view of the endoscope 6 do not move, it is possible to suppress the jaws of the medical instrument 4 outside the field of view from unintentionally coming in contact with an object such as a part in the patient's body or the like.

Further in an embodiment described above, in the state where the operation change mode is set (is turned on) through the touch panel 23, the control unit 31 changes (modifies), when it is determined that the distal end position of the medical instrument 4 is outside the field of view of the endoscope 6, the operation of the medical instrument 4 that corresponds to the command. As a result, when it is not desired to operate the medical instrument 4 because there is an aorta, vena cava, or the like near the surgical site and the medical instrument 4 cannot be visually confirmed, the operation of the medical instrument 4 outside the field of view of the endoscope 6 can be restricted by setting the operation change mode.

Further in an embodiment described above, in the state where the operation change mode is not set (is turned off) through the touch panel 23, the control unit 31 operates the medical instrument 4 in response to the command without changing (modifying) the operation of the medical instrument 4, even when the distal end position of the medical instrument 4 is located outside the field of view of the endoscope 6. Accordingly, when the influence of the contact of the medical instrument 4 with the periphery of the surgical site is small, the medical instrument 4 can be operated even outside the field of view of the endoscope 6 in the same manner as in the field of view of the endoscope 6. Therefore, the surgery can be performed efficiently.

Further in an embodiment described above, in the state where one of the first surgical sites is set (selected) through the input device 33, the control unit 31 changes (modifies), when it is determined that the distal end position of the medical instrument 4 is outside the field of view of the endoscope 6, the operation of the medical instrument 4 that corresponds to the command. Therefore, when performing surgery on one of the first surgical sites where the medical instrument 4 does not be wanted to operate when the medical instrument 4 cannot be visually confirmed, the operation of the medical instrument 4 outside the field of view of the endoscope 6 is restricted.

Further in an embodiment described above, in the state where one of the second surgical sites which are different from the first surgical sites is set (selected) through the input device 33, the control unit 31 does not change (modify), even when the distal end position of the medical instrument 4 is outside the field of view of the endoscope 6, the operation of the medical instrument 4 that corresponds to the command and thus operates the medical instrument 4 according to the command. Therefore, when performing surgery on one of the second surgical sites with a small influence on contact with the medical instrument 4, the medical instrument 4 even outside the field of view of the endoscope 6 can be operated in the same manner as in the field of view of the endoscope 6. Therefore, the surgery can be performed efficiently.

Further in an embodiment described above, the image processing device 8b is configured, when it is determined that the distal end position of the medical instrument 4 is outside the field of view of the endoscope 6, display, on the display 8a and the monitor 24, the mark MK that indicates that the medical instrument 4 is located outside the field of view of the endoscope 6. With this configuration, the operator O can easily recognize that the medical instrument 4 is located outside the field of view of the endoscope 6

Further in an embodiment described above, when it is determined that the distal end position of the medical instrument 4 is outside the field of view of the endoscope 6, the image processing device 8b displays the pull-back indicator PU that prompts to pull the endoscope 6 toward the proximal end side of the endoscope 6 (or the zoom-out indicator that prompts to zoom out) so as to lead the distal end position of the medical instrument 4 to come in the field of view of the endoscope 6. With this configuration, the operator O can easily recognize that the pulling of the endoscope 6 toward the proximal end side of the endoscope 6 leads the distal end of the medical instrument 4 to come in the field of view of the endoscope 6.

### (Modifications)

Note that one or more embodiments disclosed herein should be considered as exemplary in all respects and do not limit the invention. The scope of the invention is indicated by claims, not by explanation of one or more embodiments described above, and includes equivalents to the claims and all alterations (modification) within the same.

For example, in an embodiment described above, the case has been described in which the image processing device 8b acquires the image from the endoscope 6 and generates the graphical user interface G. However, the invention is not limited thereto. For example, the control unit 31 of the medical manipulator 1, a control unit (not illustrated) of the remote control apparatus 2, or the like may generate the graphical user interface G. Further, an image processing device that acquires the image from the endoscope 6 to perform image processing and an image processing device that generates a graphical user interface G to be superimposed on the image from the endoscope 6 may be separately provided.

Further in an embodiment described above, the case has been described in which the control unit 31 of the medical manipulator 1 is configured to determine whether or not the distal end of the instrument 4 is located outside the field of view of the endoscope 6 based on the relationship between the field of view of the endoscope 6 and the position of the distal end of the medical instrument 4. However, the invention is not limited thereto. For example, the image processing device 8b or a control unit (not illustrated) of the remote control apparatus 2 may determine whether or not the distal end of the instrument 4 is located outside the field of view of the endoscope 6 based on the relationship between the field of view of the endoscope 6 and the position of the distal end of the medical instrument 4. Further, the above determination processing may be performed by a plurality of control units (for example, the control unit 31, the image processing device 8b, the remote control device 2, etc.).

Further in an embodiment as described above, the case has been described in which the mark MK indicating that the medical instrument 4 is located outside the field of view of the endoscope 6 and including the arrow is displayed when it is determined that the distal end position of the medical instrument 4 is outside the field of view of the endoscope 6. However, the invention is not limited thereto. For example, the mark indicating that the medical instrument 4 is located outside the field of view of the endoscope 6 may be displayed without the arrow.

Further in an embodiments described above, the case has been described in which when it is determined that the distal end of the medical instrument 4 that is being operated by the operation handle 21 has come out of the field of view of the endoscope 6, the pull-back indicator PU and the mark MK are displayed on the graphical user interface G. However, the invention is not limited thereto. For example, the mark MK1 may also be displayed on the graphical user interface G when a predetermined input is made through a predetermined input device. For example, when a predetermined input is made by the camera pedal 22c or the enable switch 81 or when a predetermined input is made by a switch provided to the operation handle 21, the pull-back indicator PU and the mark MK may be displayed on the graphical user interface G.

Further in an embodiments described above, the case has been described in which the command received by the cutting pedal 22d or the coagulation pedal 22e comprises the energization command to supply the electrical energy to the medical instrument 4. However, the invention is not limited thereto. For example, a switch may be provided to the grip member 21f or the link portion 21d of the operation handle 21, so as to issue, by operating the switch, the energization command to supply the electrical energy to the medical instrument 4.

Further in an embodiments described above, the case has been described in which the operation of the surgical instrument in response to the command received through the operating device is restricted according to the set (selected) surgical site. The invention is not limited thereto. For example, types of surgery methods and types of operation restrictions corresponding to the surgery methods may be set in advance using the touch panel 23, and the operation of the surgical instrument in response to the command received through the operating device may be restricted according to a set (selected) surgery method. The setting (selection) of one of the surgery methods may be done through the input device 33, the touch panel 23, or the like.

The functions of each of the elements disclosed herein may be carried out by a circuitry or a processing circuitry including a general purpose processor, a dedicated processor, an integrated circuit, an ASIC (Application Special Integrated Circuit), a conventional circuit, or a combination of two or more of them, that is configured or programmed to perform the functions. A processor is considered a processing circuitry or a circuitry because it contains transistors and other circuit elements. In the disclosure, a circuit, a unit, or a means may be either a hardware that is configured to perform the recited function(s) or a hardware that is programmed to perform the recited function(s).

The hardware may be the hardware disclosed herein, or may be other known hardware that is programmed or configured to perform the function(s) described. If the hardware is a processor which is considered as a type of a circuit, a circuit, a means, or a unit is a combination of hardware and software, and the software is used to configure the hardware and/or the processor.

### Aspects

It may be appreciated by those skilled in the art that one or more exemplary embodiments described above may be specific examples of the following aspects.

### (Item 1)

A surgical system comprising:
a first manipulator that supports an endoscope;
a second manipulator that supports a surgical instrument;
an operating device that receives a command for operating the surgical instrument; and
a controller including one or more processors and configured to control operation of the surgical instrument in response to the command received through the operating device, wherein the controller is configured to determine, based on a relationship between a field of view of the endoscope and a position of a distal end of the surgical instrument, whether or not the distal end of the surgical instrument is located outside the field of view of the endoscope, and configured to change, when it is determined that the distal end of the surgical instrument is located outside the field of view of the endoscope, operation of the surgical instrument corresponding to the command received through the operating device from the operation of the surgical instrument in a state where the distal end of the surgical instrument is located in the field of view of the endoscope.

### (Item 2)

The surgical system according to Item 1, wherein
the controller is configured, when the distal end position of the surgical instrument changes from being outside the field of view of the endoscope to being within the field of view of the endoscope, to release the processing that changes the operation of the medical instrument that corresponds to the command received by the operating device.

### (Item 3)

The surgical system according to Item 1 or 2, wherein
the command includes a movement command of the surgical instrument to move the surgical instrument, and
the controller is configured, when it is determined that the distal end of the surgical instrument is located outside the field of view of the endoscope, control the second manipulator not to move the surgical instrument regardless of the movement command to move the surgical instrument.

### (Item 4)

The surgical system according to any one of Items 1 to 3, wherein
the command includes a movement command to move the surgical instrument,
the surgical instrument is set to move with a predetermined scaling in response to the movement command, and
the controller is configured, when it is determined that the distal end of the surgical instrument is located outside the field of view of the endoscope, control the second manipulator to move the surgical instrument with a scaling smaller than the predetermined scaling in response to the movement command to move the surgical instrument.

### (Item 5)

The surgical system according to any one of Items 1 to 4, wherein
the surgical instrument includes an electrosurgical instrument configured to supply electrical energy to the distal end of the electrosurgical instrument,
the command includes an energization command to supply the electrical energy,
the controller is configured, when it is determined that the distal end position of the surgical instrument is located outside the field of view of the endoscope, to control the electric surgical instrument not to supply the electric energy regardless of the energizing command.

### (Item 6)

The surgical system according to any one of Items 1 to 5, wherein
the surgical instrument includes jaws configured to open and close to each other,
the command includes an opening/closing command to open or close the jaws, and
the controller is configured, when it is determined that the distal end position of the surgical instrument is located outside the field of view of the endoscope, to control operation of the jaws of the surgical instrument not to open or close the jaws regardless of the opening/closing command.

### (Item 7)

The surgical system according to any one of Items 1 to 6, further comprising
a mode setting device configured to set an operation change mode, wherein
in a state where the operation change mode is turned on through the mode setting device, the controller is configured, when it is determined that the distal end position of the surgical instrument is located outside the field of view of the endoscope, to change the operation of the surgical instrument that corresponds to the command.

### (Item 8)

The surgical system according to Item 7, wherein
in a state where the operation change mode is turned off through the mode setting device, the controller is configured, when it is determined that the distal end position of the surgical instrument is located outside the field of view of the endoscope, to operate the surgical instrument according to the command without changing the operation of the surgical instrument that corresponds to the command.

### (Item 9)

The surgical system according to any one of Items 1 to 8, further comprising
a surgical site input device configured to input a surgical site, wherein
in a state where a first surgical site is set through the surgical site input device, the controller is configured, when it is determined that the distal end position of the surgical instrument is located outside the field of view of the endoscope, to change the operation of the surgical instrument that corresponds to the command.

### (Item 10)

The surgical instrument according to Item 9, wherein
in a state where a second surgical site which is different from the first surgical site is set through the input device, the controller is configured not to change the operation of the surgical instrument that corresponds to the command and thus operate the surgical instrument according to the command, even when the distal end position of the surgical instrument is outside the field of view of the endoscope.

### (Item 11)

The surgical instrument according to Item 10, further comprising
a display device configured to display an image captured by the endoscope, wherein
the controller is configured, when it is determined that the distal end of the surgical instrument is located outside the field of view of the endoscope, to display on the display device a first indicator that indicates the surgical instrument is located outside the field of view of the endoscope.

### (Item 12)

The surgical system according to Item 11, wherein
the controller is configured, when it is determined that the distal end position of the surgical instrument is outside the field of view of the endoscope, to display a second indicator that prompts to pull back the endoscope toward a proximal end side of the endoscope so as to lead the distal end position of the surgical instrument to come in the field of view of the endoscope.

### (Item 13)

The surgical system according to any one of Items 1 to 12, wherein
the operating device is provided at the second manipulator.

### (Item 14)

The surgical system according to any one of Items 1 to 12, wherein
the operating device is provided independently from the first manipulator and the second manipulator.

### (Item 15)

A method of operating a surgical system, comprising:
determining, based on a relationship between a field of view of an endoscope supported by a first manipulator and a position of a distal end of a surgical instrument supported by a second manipulator, whether or not a distal end of the surgical instrument is located outside the field of view of the endoscope; and
when the distal end of the surgical instrument is located outside the field of view of the endoscope, changing an operation of the surgical instrument that corresponds to the command received through an operating device from a first operation mode of the surgical instrument for a state where the distal end of the surgical instrument is located in the field of view of the endoscope.

The invention includes other embodiments or modifications in addition to one or more embodiments described above without departing from the spirit of the invention. The one or more embodiments described herein are to be considered in all respects as illustrative, and not restrictive. The scope of the invention is indicated by the appended claims rather than by the foregoing description. Hence, all configurations including the meaning and range within equivalent arrangements of the claims are intended to be embraced in the invention.

1. A surgical system comprising:
a first manipulator that supports an endoscope;
a second manipulator that supports a surgical instrument;
an operating device configured to receive a command to operate the surgical instrument; and
a controller including one or more processors and configured to control operation of the surgical instrument in a first operation mode that controls the operation of the surgical instrument in response to the command received through the operating device, wherein
the controller is configured to determine, based on a relationship between a field of view of the endoscope and a position of a distal end of the surgical instrument, whether or not the distal end of the surgical instrument is located outside the field of view of the endoscope, when it is determined that the distal end of the surgical instrument is located inside the field of view of the endoscope, control the operation of the surgical instrument in the first operation mode, and when it is determined that the distal end of the surgical instrument is located outside the field of view of the endoscope, change the operation of the surgical instrument from the first operation mode to a second operation mode.

2. The surgical system according to claim 1, wherein
the controller is configured, when the distal end position of the surgical instrument changes from being outside the field of view of the endoscope to being within the field of view of the endoscope, to release the second operation mode that changes the operation of the surgical instrument that corresponds to the command received by the operating device.

3. The surgical system according to claim 1, wherein
the command includes a movement command to move the surgical instrument, and
the controller is configured, under the second operation mode, control the second manipulator not to move the surgical instrument regardless of the movement command.

4. The surgical system according to claim 1, wherein
the command includes a movement command to move the surgical instrument,
the surgical instrument is set to move with a predetermined scaling in response to the movement command, and
the controller is configured, under the second operation mode, control the second manipulator to move the surgical instrument with a scaling smaller than the predetermined scaling in response to the movement command to move the surgical instrument.

5. The surgical system according to claim 1, wherein
the surgical instrument comprises an electrosurgical instrument configured to supply electrical energy to the distal end of the electrosurgical instrument,
the command includes an energization command to supply the electrical energy,
the controller is configured, under the second operation mode, to control the electrosurgical instrument not to supply the electrical energy regardless of the energizing command.

6. The surgical system according to claim 1, wherein
the surgical instrument includes jaws configured to open and close to each other,
the command includes an opening/closing command to open or close the jaws, and
the controller is configured, under the second operation mode, to control the jaws of the surgical instrument not to open or close the jaws of the surgical instrument regardless of the opening/closing command.

7. The surgical system according to claim 1, further comprising
a mode setting device configured to set an operation change mode, wherein
in a state where the operation change mode is turned on through the mode setting device, the controller is configured to determine whether or not the distal end position of the surgical instrument is located outside the field of view of the endoscope, and configured, when it is determined that the distal end position of the surgical instrument is located outside the field of view of the endoscope, to change the operation of the surgical instrument from the first operation mode to the second operation mode that changes the operation of the surgical instrument that corresponds to the command.

8. The surgical system according to claim 7, wherein
in a state where the operation change mode is turned off through the mode setting device, the controller is configured, even when the distal end position of the surgical instrument is located outside the field of view of the endoscope, to not change from the first operation mode to the second operation mode, thereby to operate the surgical instrument according to the command without changing the operation of the surgical instrument that corresponds the command.

9. The surgical system according to claim 1, further comprising
a surgical site input device configured to input a surgical site, wherein
in a state where a first surgical site is set through the surgical site input device, the controller is configured to determine whether or not the distal end position of the surgical instrument is located outside the field of view of the endoscope, and configured, when it is determined that the distal end position of the surgical instrument is located outside the field of view of the endoscope, to change the operation of the surgical instrument from the first operation mode to the second operation mode that changes the operation of the surgical instrument that corresponds to the command.

10. The surgical system according to claim 9, wherein
in a state where a second surgical site which is different from the first surgical site is set through the surgical site input device, the controller is configured, even when the distal end position of the surgical instrument is outside the field of view of the endoscope, to not change the operation of the surgical instrument from the first operation mode to the second operation mode, thereby to operate the surgical instrument according to the command without changing the operation of the surgical instrument that corresponds to the command.

11. The surgical system according to claim 1, further comprising
a display device configured to display an image captured by the endoscope, wherein
the controller is configured, when it is determined that the distal end of the surgical instrument is located outside the field of view of the endoscope, to display a first indicator that indicates the surgical instrument is located outside the field of view of the endoscope.

12. The surgical system according to claim 11, wherein
the controller is configured, when it is determined that the distal end position of the surgical instrument is outside the field of view of the endoscope, to display a second indicator that prompts to pull back the endoscope toward a proximal end side of the endoscope so as to lead the distal end position of the surgical instrument to come in the field of view of the endoscope.

13. The surgical system according to claim 1, wherein
the operating device is provided at the second manipulator.

14. The surgical system according to claim 1, wherein
the operating device is provided independently from the first manipulator and the second manipulator.

15. A surgical system comprising:
a first manipulator that supports an endoscope;
a second manipulator that supports a surgical instrument;
an operating device configured to receive a command to operate the surgical instrument;
a mode setting device configured to set an operation change mode;
a controller including one or more processors and configured to control operation of the surgical instrument in response to the command received through the operating device;
the controller is configured to:
operate the surgical instrument according to the command in a state where the operation change mode is turned off through the mode setting device; and
change the operation of the surgical instrument that corresponds to the command, when it is determined that the distal end position of the surgical instrument is located outside a field of view of the endoscope in a state where the operation change mode is turned on through the mode setting device.

16. The surgical system according to claim 15, wherein
in a state where the operation change mode is turned on through the mode setting device, the controller is configured, when it is determined that the distal end position of the surgical instrument is located inside the field of view of the endoscope, to operate the surgical instrument according to the command.

17. A surgical system comprising:
a first manipulator that supports an endoscope;
a second manipulator that supports a surgical instrument;
an operating device configured to receive a command to operate the surgical instrument;
a surgical site input device configured to input a surgical site;
a controller including one or more processors and configured to control operation of the surgical instrument according to the command received through the operating device;
the controller is configured to:
change the operation of the surgical instrument that corresponds to the command, when it is determined that a distal end of the surgical instrument is located outside a field of view of the endoscope in a state where a first surgical site is set through the surgical site input device; and
operate the surgical instrument according to the command, in a state where a second surgical site different from the first surgical site is set through the surgical site input device.

18. The surgical system according to claim 17, wherein
in a state where the first surgical site is set through the surgical site input device, the controller is configured, when it is determined that the distal end of the surgical instrument is located inside the field of view of the endoscope, to operate the surgical instrument according to the command.

19. The controller according to claim 17, wherein
the command includes a movement command to move the surgical instrument, and
in a state where the first surgical site is set through the surgical site input device, the controller is configured, when it is determined that the distal end of the surgical instrument is located inside the field of view of the endoscope, to control the second manipulator to move the surgical instrument according to the movement command, and is configured, when it is determined that the distal end of the surgical instrument is located outside the field of view of the endoscope, to control the second manipulator not to move the surgical instrument even with receiving the movement command.

20. A method of operating a surgical instrument by a controller that is included in a surgical system that includes a first manipulator that supports an endoscope, a second manipulator that supports the surgical instrument, and an operating device configured to receive a command to operate the surgical instrument, the method comprising:
determining whether or not a distal end of the surgical instrument is located outside a field of view of the endoscope based on a relationship between a position of the distal end of the surgical instrument and the field of view of the endoscope;
when it is determined that the distal end of the surgical instrument is located inside the field of view of the endoscope, controlling operation of the surgical instrument in a first operation mode that controls the operation of the surgical instrument in response to the command received through the operating device; and
when it is determined that the distal end of the surgical instrument is located outside the field of view of the endoscope, changing from the first operation mode to a second operation mode, and controlling the operation of the surgical instrument in the second operation mode.
